SEQUENCE LISTING (14 sequences)

SEQ ID NO: 1 - protein, 735 aa, Homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRR

SEQ ID NO: 2 - protein, 14 aa, homo sapiens
LGLYNLMNITRGSV

SEQ ID NO: 3 - protein, 13 aa, homo sapiens
KGKTNCPATVING

SEQ ID NO: 4 - protein, 13 aa, homo sapiens
WSKHNLTITQGKL

SEQ ID NO: 5 - protein, 20 aa, homo sapiens
NVTEFDGQDACGSNSWTVVD

SEQ ID NO: 6 - protein, 10 aa, homo sapiens
DIIYVQTDAT

SEQ ID NO: 7 - protein, 18 aa, homo sapiens
YEDSAGECCSCPKTDSQI

SEQ ID NO: 8 - protein, 620 aa, homo sapiens
SLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 9 - protein, 10 aa, homo sapiens
KKNGRILTLP

SEQ ID NO: 10 - protein, 11 aa, homo sapiens
RILTLPRSNPS

SEQ ID NO: 11 - protein, 7 aa, homo sapiens
KNGRILT

SEQ ID NO: 12 - protein, 17 aa, homo sapiens
GGKKNGRILTLPRSNPS

SEQ ID NO: 13 - protein, 18 aa, homo sapiens
NGGKKNGRILTLPRSNPS

SEQ ID NO: 14 - protein, 1382 aa, homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

(19) United States
(12) Patent Application Publication
(10) Pub. No.: US 2016/0009809 A1
(43) Pub. Date: Jan. 14, 2016

(54) VETERINARY COMPOSITION AND METHOD OF IMPROVING LIVABILITY OF ANIMALS, PROMOTING LIVE WEIGHT GAIN IN MAMMALS AND BIRDS, ENHANCING THE EFFECTIVENESS OF IMMUNIZATION, AND PREVENTING AND/OR TREATING INFECTIOUS DISEASES

(71) Applicant: Oleg Iliich Epshtein, Moscow (RU)

(72) Inventor: Oleg Iliich Epshtein, Moscow (RU)

(21) Appl. No.: 14/730,775

(22) Filed: Jun. 4, 2015

(30) Foreign Application Priority Data
Jun. 6, 2014 (RU) 2014123129

(51) Int. Cl. C07K 16/28

(52) U.S. Cl. CPC C07K 16/2869; A61K 2039/505

(57) ABSTRACT

The present application provides a veterinary composition comprising an activated-potentiated form of an antibody to insulin receptor which can be used for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

This application claims priority to Russian Patent Application No.: 2014123129, filed Jun. 6, 2014, all of which is hereby incorporated by reference in its entirety.

### FIELD

This invention relates to veterinary medicine and is useful for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiologies), and increasing livestock performance, reproduction and survival.

### BACKGROUND

Over past few decades, the world's meat producing industry has been undergoing brisk, spasmodic changes in search of the ways to meet the growing consumer demand. Alongside with this, scientific interest in organic production of mammalian and poultry meat has increased immensely.

Livestock and poultry breeding industry relies on a wide use of non-nutritional food supplements, primarily antibiotics, in order to improve performance and immune status of animals. Some of these supplements are indicated for chemotherapeutic and prophylactic purposes, whereas others are employed as growth promoters.

Prolonged use of feeds supplemented with subtherapeutic doses of such additives may result in an accumulation of their residuals in animal-derived products and development of drug-resistant microorganisms in humans.

The use of antibiotics as the pivotal part of breeding programmes has recently been abandoned by most poultry and mammalian meat producers. The EU has issued a recommendation against the use of antibiotics, including chlortetracycline, as growth stimulants and means to enhance production efficiency and reduce livestock mortality (Perreten V. 2003 Use of antimicrobials in food producing animals in Switzerland and the European Union (EU). Mitt. Lebensm. Hyg. 94:155-163). This is justified by the fact that the resistance of microorganisms to antibiotics and their fragments in meat products may be detrimental to users' health. The ban on synthetic feed supplements has spawned high-profile research and investigational development of alternative animal health and performance enhancers that could meet the needs of continuously evolving meat industry. The most important selection efforts are focused on growth promotion, though such interferences have been found to negatively correlate with the immune status of animals and poultry. Most investigations are now dealing with the issue of designing new medicines that could be used as growth promoters in the husbandry of both mammals and birds and enhance livestock performance and immunological resistance to numerous diseases. Growth promoters, such as probiotics, prebiotics and immunomodulators, were developed as an alternative to antibiotic growth stimulants. For such agents it has been shown that mammalian and bird species that are genetically characterized by a large body size are able to elicit a far less prominent humoral immune response (Miller L. L., Siegel P. B., and Dunnington E. A. 1992. Inheritance of antibody response to sheep erythrocytes in lines of chickens divergently selected for fifty-six-day body weight and their crosses. Poult. Sci., 71: 47-52).

There are veterinary drug compositions known in the art that are used for the prevention/treatment of a large number of diseases, including infectious ones (RU 20059408 CI, A61K9/08, 1996; RU 2440121 C1, A61K31/7016, 2011).

Also, there is a range of plant-derived food supplements, known in the art, including different microelements, ferments and synthetic compounds (RU 2007456 C1, A23K1/65, 1994; RU 2105496 C1, A23K1/16, 1998; RU 2340204 C1, A23K1/00, 2008; RU 2420089 C1, A23K1/00, 2011; RU 2450532 C1, A23K1/00, 2012), added in large amounts to animal feed rations.

In addition, there are growth promoters, known in the art, used to increase body weight gain in animals (RU 2102063 C1, A23K1/00, 1998; RU 2268043 C2, A23K31/41, 2006; I. F. KLENOVA, N. A. YAREMENKO. Veterinary Drugs in Russia, Guide. Moscow, Sel'khozizdat, 2001, P. 171-174; N. V. DEMIDOV. Anthelmintics in Veterinary Practice. Moscow, “Kolos” Publisher, 1982, P. 250-298).

However, the abovementioned drugs generally have a limited efficacy range and may cause adverse effects.

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of diseases of viral etiology. See U.S. Pat. No. 7,572,441, which is incorporated herein by reference in its entirety.

The present invention is directed to an effective and safe veterinary composition and methods of its use for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), increasing animal welfare and increasing livestock performance, reproduction and survival.

The solution to the existing problem is presented in form of a veterinary composition, which comprises an activated-potentiated form of antibodies to insulin receptor.

### SUMMARY

In one aspect, the invention provides a method of improving livability of food-producing animals, non-human mammals and birds, said method comprising administering to the animal, non-human mammal or bird an activated-potentiated form of an antibody to the insulin receptor. Preferably, the method of improving livability of food-producing animals (mammals and birds) involves administering to an animal an activated-potentiated form of an antibody to the insulin receptor β-subunit or to a C-terminal fragment of the insulin receptor β-subunit. In an embodiment, a mixture of various homeopathic dilutions of an antibody to a C-terminal fragment of the insulin receptor β-subunit is used as a unit dosage form.

Particularly contemplated is a variant of this aspect comprising administration of activated-potentiated form of an antibody to a C-terminal fragment of the insulin receptor β-subunit, wherein said activated-potentiated form is represented by an aqueous or aqueous-alcoholic solution with the activity achieved through repeated sequential dilution of the primary matrix solution of the antibody to a C-terminal fragment of the insulin receptor β-subunit in a water or alcohol-water solvent, coupled with external mechanical treatment.

In another aspect, the invention provides a method of promoting body weight gain in non-human mammals or birds, said method comprising administering to the non-human mammal or bird an activated-potentiated form of an antibody to the insulin receptor, preferably to the insulin receptor β-subunit.

Particularly contemplated is a variant of this aspect comprising administration of activated-potentiated form of an antibody to a C-terminal fragment of the insulin receptor β-subunit, wherein said activated-potentiated form is represented by an aqueous or aqueous-alcoholic solution with the activity achieved through repeated sequential dilution of the primary matrix solution of the antibody to a C-terminal fragment of the insulin receptor β-subunit in a water or alcohol-water solvent, coupled with external mechanical treatment of each dilution.

In accordance with this aspect of the invention, a mixture of various homeopathic dilutions of an antibody to a C-terminal fragment of the insulin receptor β-subunit is used as a unit dosage form.

In another aspect, the invention provides a method of enhancing the effectiveness of immunization in non-human mammals or birds, said method comprising administering to the non-human mammal or bird an activated-potentiated form of an antibody to insulin receptor, preferably to the insulin receptor β-subunit. In an embodiment, a mixture of various homeopathic dilutions of an antibody to a C-terminal fragment of the insulin receptor β-subunit is used as a unit dosage form.

Particularly contemplated is a variant of this aspect comprising administration of activated-potentiated form of an antibody to a C-terminal fragment of the insulin receptor β-subunit, wherein said activated-potentiated form is represented by an activated-potentiated aqueous or aqueous-alcoholic solution with the activity achieved through repeated sequential dilution of the primary (matrix) solution of the antibody to a C-terminal fragment of the insulin receptor β-subunit in a water or alcohol-water solvent, coupled with external mechanical treatment of each dilution.

In another aspect, the invention provides a method of preventing and/or treating infectious diseases of mammals and birds, said method comprising administering to an animal an activated-potentiated form of an antibody to the insulin receptor, preferably an antibody to the insulin receptor β-subunit.

Particularly contemplated is a variant of this aspect comprising administration of activated-potentiated form of an antibody to a C-terminal fragment of the insulin receptor β-subunit, wherein said activated-potentiated form is represented by an aqueous or aqueous-alcoholic solution with the activity achieved through repeated sequential dilution of the primary matrix solution of the antibody to a C-terminal fragment of the insulin receptor β-subunit in a water or alcohol-water solvent, coupled with external mechanical treatment of each dilution.

It is particularly contemplated that a single unit dosage form incorporates a mixture of dilutions of said antibody to a C-terminal fragment of the insulin receptor β-subunit obtained according to a homeopathic manufacturing methodology.

In accordance with the invention, the maximum beneficial effect on the livability of food-producing animals, mammals and birds may be achieved through regular, long-term administration of the veterinary composition.

As a method of promoting body weight gain in mammals and birds, the veterinary composition is administered throughout the fattening period, from the first to the last day of life.

For the purposes of increasing stock performance and survival, preventing infectious diseases and enhancing the effectiveness of immunization, the veterinary composition is preferably administered for a total of three/four 4-7-day periods.

In accordance with the invention, the claimed aqueous or aqueous-alcoholic solutions have pronounced activity (potency) acquired during the treatment process involving sequential decrease in the concentration of the initial substance—antibodies to the insulin receptor β-subunit (C-terminal fragment of insulin receptor β-subunit).

In the proposed aspects of use, the activated-potentiated form of an antibody to the insulin receptor β-subunit (C-terminal fragment of insulin receptor β-subunit) broadens the range of compounds for improving animals' livability, promoting body weight gain in mammals and birds, enhancing the effectiveness of immunization, and preventing and/or treating infectious diseases, with high survival rate provided in mammals and birds. In said aspects of use, the invention produces neither adverse effects nor general toxicity or immunotoxicity effects, causes no local irritation or allergic sensitization and has no reproductive toxicity (which is attributed to the virtual absence of or ultra-low molecular concentration of the highly diluted initial substance). A long-term administration of the veterinary composition is not associated with adverse events such as hypoglycemia or acidosis. Particularly contemplated is administration of the claimed veterinary composition in combination with other bioactive feed supplements and/or drug products used both for promoting body weight gain and growth of food-producing animals, enhancing the effectiveness of immunization, and treating and/or preventing infectious diseases.

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)2, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, “homeopathic potentization” may involve, for example, repeated consecutive dilutions combined with external treatment, particularly mechanical shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term “activated-potentiated” form used in the claims.

In other words, an antibody is in the “activated-potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, “homeopathic potentization” may involve, for example, repeated consecutive dilutions combined with external treatment, particularly vertical mechanical shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated potentiated” form of the antibodies described herein is described in more detail below.

There has been a considerable amount of controversy regarding homeopathic treatment. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models.

Also, the claimed “activated-potentiated” form of antibody encompass only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated” form of the antibodies of the present invention is not attributable to the initial molecular form of the antibody. Preferred is the “activated-potentiated” form of antibody in liquid or solid form in which the concentration of the initial molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the initial molecular form of the antibody is below the Avogadro number. In pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides the methods for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

The pharmaceutical composition in accordance with this aspect of the invention may be in the liquid form or in solid form. Each of the activated potentiated forms of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R.-2005-Vol. 14.-N 1-2. P. 33-55, both incorporated herein by reference.

Monoclonal antibodies may be obtained, e.g., by means of hybridoma technology. The initial stage of the process includes immunization based on the principles already developed in course of polyclonal antisera preparation. Further stages of work involve production of hybrid cells generating clones of antibodies with identical specificity. Their separate isolation is performed using the same methods as in case of polyclonal antisera preparation.

Polyclonal antibodies may be obtained via active immunization of animals. For this purpose, for example, suitable animals (e.g. rabbits) receive a series of injections of the appropriate antigen. The animals' immune system generates corresponding antibodies, which are collected from the animals in a known manner. This procedure enables preparation of a monospecific antibody-rich serum. If desired, the serum containing antibodies may be purified, e.g., using affine chromatography, fractionation by salt precipitation, or ion-exchange chromatography. The resulting purified, antibody-enriched serum may be used as a starting material for preparation of the activated-potentiated form of the antibodies. The preferred concentration of the resulting initial solution of antibody in the solvent, preferably, water or water-ethyl alcohol mixture, ranges from about 0.5 to about 5.0 mg/ml.

The preferred procedure for preparing each component is the use of the mixture of three aqueous-alcohol dilutions of the primary matrix solution of antibodies diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In the preferred embodiment, the starting material for the preparation of the activated potentiated form that comprise the combination of the invention is polyclonal, animal-raised antibody to the corresponding antigen, namely, C-terminal fragment of beta subunit of human insulin receptor or insulin receptor. To obtain the activated-potentiated form of polyclonal antibodies to C-terminal fragment of beta subunit of human insulin receptor, the desired antigen may be injected as immunogen into a laboratory animal, preferably, rabbits′. Peptides of particular interest may include at least about 3 amino acids, usually at least about 10 on either side of the sequence, preferably having at least 3 amino acids at the C-terminal side. The following sequences of human insulin receptor are specifically contemplated as suitable antigens:

Entire alpha-subunit of human insulin receptor:

```
SEQ ID NO: 1
His Leu Tyr
  28 30
Pro Gly Glu Val Cys Pro Gly Met Asp Ile Arg Asn
  31 35 40
Asn Leu Thr Arg Leu His Glu Leu Glu Asn Cys Ser
                  45 46 50
Val Ile Glu Gly His Leu Gln Ile Leu Leu Met Phe
  55 60 61 65
Lys Thr Arg Pro Glu Asp Phe Arg Asp Leu Ser Phe
                        70 75 76
Pro Lys Leu Ile Met Ile Thr Asp Tyr Leu Leu Leu
        80 85 90
Phe Arg Val Tyr Gly Leu Glu Ser Leu Lys Asp Leu
91 95 100
Phe Pro Asn Leu Thr Val Ile Arg Gly Ser Arg Leu
                105 106 110
Phe Phe Asn Tyr Ala Leu Val Ile Phe Glu Met Val
115 120 121 125
His Leu Lys Glu Leu Gly Leu Tyr Asn Leu Met Asn
                        130 135 136
Ile Thr Arg Gly Ser Val Arg Ile Glu Lys Asn Asn
        140 145 150
Glu Leu Cys Tyr Leu Ala Thr Ile Asp Trp Ser Arg
151 155 160
Ile Leu Asp Ser Val Glu Asp Asn Tyr Ile Val Leu
                165 166 170
Asn Lys Asp Asp Asn Glu Glu Cys Gly Asp Ile Cys
175 180 181 185
Pro Gly Thr Ala Lys Gly Lys Thr Asn Cys Pro Ala
                        190 195 196
Thr Val Ile Asn Gly Gln Phe Val Glu Arg Cys Trp
        200 205 210
Thr His Ser His Cys Gln Lys Val Cys Pro Thr Ile
211 215 220
Cys Lys Ser His Gly Cys Thr Ala Glu Gly Leu Cys
                225 226 230
Cys His Ser Glu Cys Leu Gly Asn Cys Ser Gln Pro
235 240 241 245
Asp Asp Pro Thr Lys Cys Val Ala Cys Arg Asn Phe
                        250 255 256
Tyr Leu Asp Gly Arg Cys Val Glu Thr Cys Pro Pro
        260 265 270
Pro Tyr Tyr His Phe Gln Asp Trp Arg Cys Val Asn
271 275 280
Phe Ser Phe Cys Gln Asp Leu His His Lys Cys Lys
                285 286 290
Asn Ser Arg Arg Gln Gly Cys His Gln Tyr Val Ile
295 300 301 305
His Asn Asn Lys Cys Ile Pro Glu Cys Pro Ser Gly
                        310 315 316
Tyr Thr Met Asn Ser Ser Asn Leu Leu Cys Thr Pro
        320 325 330
Cys Leu Gly Pro Cys Pro Lys Val Cys His Leu Leu
331 335 340
Glu Gly Glu Lys Thr Ile Asp Ser Val Thr Ser Ala
                345 346 350
Gln Glu Leu Arg Gly Cys Thr Val Ile Asn Gly Ser
355 360 361 365
Leu Ile Ile Asn Ile Arg Gly Gly Asn Asn Leu Ala
                        370 375 376
Ala Glu Leu Glu Ala Asn Leu Gly Leu Ile Glu Glu
        380 385 390
Ile Ser Gly Tyr Leu Lys Ile Arg Arg Ser Tyr Ala
391 395 400
Leu Val Ser Leu Ser Phe Phe Arg Lys Leu Arg Leu
                405 406 410
Ile Arg Gly Glu Thr Leu Glu Ile Gly Asn Tyr Ser
415 420 421 425
Phe Tyr Ala Leu Asp Asn Gln Asn Leu Arg Gln Leu
                        430 435 436
Trp Asp Trp Ser Lys His Asn Leu Thr Ile Thr Gln
        440 445 450
Gly Lys Leu Phe Phe His Tyr Asn Pro Lys Leu Cys
451 455 460
Leu Ser Glu Ile His Lys Met Glu Glu Val Ser Gly
                465 466 470
Thr Lys Gly Arg Gln Glu Arg Asn Asp Ile Ala Leu
475 480 481 485
Lys Thr Asn Gly Asp Gln Ala Ser Cys Glu Asn Glu
                        490 495 496
Leu Leu Lys Phe Ser Tyr Ile Arg Thr Ser Phe Asp
        500 505 510
Lys Ile Leu Leu Arg Trp Glu Pro Tyr Trp Pro Pro
511 515 510
Asp Phe Arg Asp Leu Leu Gly Phe Met Leu Phe Tyr
                525 526 530
Lys Glu Ala Pro Tyr Gln Asn Val Thr Glu Phe Asp
535 540 541 545
Gly Gln Asp Ala Cys Gly Ser Asn Ser Trp Thr Val
                        550 555 556
Val Asp Ile Asp Pro Pro Leu Arg Ser Asn Asp Pro
        560 565 570
Lys Ser Gln Asn His Pro Gly Trp Leu Met Arg Gly
571 575 580
Leu Lys Pro Trp Thr Gln Tyr Ala Ile Phe Val Lys
                585 586 590
Thr Leu Val Thr Phe Ser Asp Glu Arg Arg Thr Tyr
595 600 601 605
Gly Ala Lys Ser Asp Ile Ile Tyr Val Gln Thr Asp
                        610 615 616
Ala Thr Asn Pro Ser Val Pro Leu Asp Pro Ile Ser
        620 625 630
Val Ser Asn Ser Ser Ser Gln Ile Ile Leu Lys Trp
631 635 640
Lys Pro Pro Ser Asp Pro Asn Gly Asn Ile Thr His
                645 646 650
Tyr Leu Val Phe Trp Glu Arg Gln Ala Glu Asp Ser
655 660 661 665
Glu Leu Phe Glu Leu Asp Tyr Cys Leu Lys Gly Leu
                        670 675 676
Lys Leu Pro Ser Arg Thr Trp Ser Pro Pro Phe Glu
        680 685 690
Ser Glu Asp Ser Gln Lys His Asn Gln Ser Glu Tyr
691 695 700
Glu Asp Ser Ala Gly Glu Cys Cys Ser Cys Pro Lys
                705 706 710
Thr Asp Ser Gln Ile Leu Lys Glu Leu Glu Glu Ser
715 720 721 725
Ser Phe Arg Lys Thr Phe Glu Asp Tyr Leu His Asn
                        730 735 736
Val Val Phe Val Pro Arg Lys Thr Ser Ser Gly Thr
        740 745 750
Gly Ala Glu Asp Pro Arg Pro Ser Arg Lys Arg Arg
751 755 760 762
```

### Fragments of Alpha-Subunit of Human Insulin Receptor:

```
SEQ ID NO: 2
Leu Gly Leu Tyr Asn Leu Met Asn Ile Thr Arg
131 135 136 140
Gly Ser Val
                144
SEQ ID NO: 3
Lys Gly Lys Thr Asn Cys Pro Ala Thr Val Ile
191 195 196 200
Asn Gly
        203
SEQ ID NO: 4
Trp Ser Lys His Asn Leu Thr Ile Thr Gln Gly
441 445 450 451
Lys Leu
        453
SEQ ID NO: 5
Asn Val Thr Glu Phe Asp Gly Gln Asp Ala Cys
541 545 550
Gly Ser Asn Ser Trp Thr Val Val Asp
                        555 556 560
SEQ ID NO: 6
Asp Ile Ile Tyr Val Gln Thr Asp Ala Thr
611 615 616 620
SEQ ID NO: 7
Tyr Glu Asp Ser Ala Gly Glu Cys Cys Ser Cys
702 705 706 710
Pro Lys Thr Asp Ser Gln Ile
                715 719
```

### Entire Beta Subunit of Human Insulin Receptor:

```
SEQ ID NO: 8
Ser Leu Gly Asp Val Gly Asn Val Thr Val Ala Val
763 765 766 770
Pro Thr Val Ala Ala Phe Pro Asn Thr Ser Ser Thr
775 780 781 785
Ser Val Pro Thr Ser Pro Glu Glu His Arg Pro Phe
                        790 795 796
Glu Lys Val Val Asn Lys Glu Ser Leu Val Ile Ser
        800 805 810
Gly Leu Arg His Phe Thr Gly Tyr Arg Ile Glu Leu
811 815 820
Gln Ala Cys Asn Gln Asp Thr Pro Glu Glu Arg Cys
                825 826 830
Ser Val Ala Ala Tyr Val Ser Ala Arg Thr Met Pro
835 840 841 845
Glu Ala Lys Ala Asp Asp Ile Val Gly Pro Val Thr
                        850 855 856
His Glu Ile Phe Glu Asn Asn Val Val His Leu Met
        860 865 870
Trp Gln Glu Pro Lys Glu Pro Asn Gly Leu Ile Val
871 875 880
Leu Tyr Glu Val Ser Tyr Arg Arg Tyr Gly Asp Glu
                885 886 890
Glu Leu His Leu Cys Val Ser Arg Lys His Phe Ala
895 900 901 905
Leu Glu Arg Gly Cys Arg Leu Arg Gly Leu Ser Pro
                        910 915 916
Gly Asn Tyr Ser Val Arg Ile Arg Ala Thr Ser Leu
        920 925 930
Ala Gly Asn Gly Ser Trp Thr Glu Pro Thr Tyr Phe
931 935 940
Tyr Val Thr Asp Tyr Leu Asp Val Pro Ser Asn Ile
                945 946 950
Ala Lys Ile Ile Ile Gly Pro Leu Ile Phe Val Phe
955 960 961 965
Leu Phe Ser Val Val Ile Gly Ser Ile Tyr Leu Phe
                        970 975 976
Leu Arg Lys Arg Gln Pro Asp Gly Pro Leu Gly Pro
        980 985 990
Leu Tyr Ala Ser Ser Asn Pro Glu Tyr Leu Ser Ala
991 995 1000
Ser Asp Val Phe Pro Cys Ser Val Tyr Val Pro Asp
              1005 1006 1010
Glu Trp Glu Val Ser Arg Glu Lys Ile Thr Leu Leu
1015 1020 1021 1025
Arg Glu Leu Gly Gln Gly Ser Phe Gly Met Val Tyr
                      1030 1035 1036
Glu Gly Asn Ala Arg Asp Ile Ile Lys Gly Glu Ala
      1140 1145 1050
Glu Thr Arg Val Ala Val Lys Thr Val Asn Glu Ser
1051 1155 1160
Ala Ser Leu Arg Glu Arg Ile Glu Phe Leu Asn Glu
              1065 1066 1170
  Ala Ser Val Met Lys Gly Phe Thr Cys His His Val
1175 1080 1081 1185
Val Arg Leu Leu Gly Val Val Ser Lys Gly Gln Pro
                      1190 1095 1096
Thr Leu Val Val Met Glu Leu Met Ala His Gly Asp
      1100 1105 1110
Leu Lys Ser Tyr Leu Arg Ser Leu Arg Pro Glu Ala
1111 1115 1120
Glu Asn Asn Pro Gly Arg Pro Pro Pro Thr Leu Gln
              1125 1126 1130
  Glu Met Ile Gln Met Ala Ala Glu Ile Ala Asp Gly
1135 1140 1141 1145
Met Ala Tyr Leu Asn Ala Lys Lys Phe Val His Arg
                      1150 1155 1156
Asp Leu Ala Ala Arg Asn Cys Met Val Ala His Asp
      1160 1165 1170
Phe Thr Val Lys Ile Gly Asp Phe Gly Met Thr Arg
1171 1175 1180
Asp Ile Tyr Glu Thr Asp Tyr Tyr Arg Lys Gly Gly
              1185 1186 1190
  Lys Gly Leu Leu Pro Val Arg Trp Met Ala Pro Glu
1195 1200 1201 1205
Ser Leu Lys Asp Gly Val Phe Thr Thr Ser Ser Asp
                      1210 1215 1216
Met Trp Ser Phe Gly Val Val Leu Trp Glu Ile Thr
      1220 1225 1230
Ser Leu Ala Glu Gln Pro Tyr Gln Gly Leu Ser Asn
1231 1235 1240
Glu Gln Val Leu Lys Phe Val Met Asp Gly Gly Tyr
              1245 1246 1250
  Leu Asp Gln Pro Asp Asn Cys Pro Glu Arg Val Thr
1255 1260 1261 1265
Asp Leu Met Arg Met Cys Trp Gln Phe Asn Pro Lys
                      1270 1275 1276
Met Arg Pro Thr Phe Leu Glu Ile Val Asn Leu Leu
      1280 1285 1290
Lys Asp Asp Leu His Pro Ser Phe Pro Glu Val Ser
1291 1295 1300
Phe Phe His Ser Glu Glu Asn Lys Ala Pro Glu Ser
              1305 1306 1310
  Glu Glu Leu Glu Met Glu Phe Glu Asp Met Glu Asn
1315 1320 1321 1325
Val Pro Leu Asp Arg Ser Ser His Cys Gln Arg Glu
                      1330 1335 1336
Glu Ala Gly Gly Arg Asp Gly Gly Ser Ser Leu Gly
      1340 1345 1350
Phe Lys Arg Ser Tyr Glu Glu His Ile Pro Tyr Thr
1351 1355 1360
His Met Asn Gly Gly Lys Lys Asn Gly Arg Ile Leu
              1365 1366 1370
  Thr Leu Pro Arg Ser Asn Pro Ser
1375 1380 13811382
```

### Fragments of C-Terminal Fragment of Beta Subunit of Human Insulin Receptor:

```
SEQ ID NO: 9
Lys Lys Asn Gly Arg Ile Leu Thr Leu Pro
1368 1370 1375 1377
SEQ ID NO: 10
  Arg Ile Leu Thr Leu Pro Arg Ser Asn
1372 1375 1380
Pro Ser
13811382
SEQ ID NO: 11
Lys Asn Gly Arg Ile Leu Thr
13691370 1375
SEQ ID NO: 12
Gly Gly Lys Lys Asn Gly Arg Ile Leu Thr Leu Pro
1366 1370 1375
Arg Ser Asn Pro Ser
              1380 13811382
SEQ ID NO: 13
  Asn Gly Gly Lys Lys Asn Gly Arg Ile Leu Thr Leu
1365 1366 1370 1375
Pro Arg Ser Asn Pro Ser
                      1380 13811382
```

The use of human insulin receptor as antigen is also contemplated. The suitable sequence for such antigen is as follow:

```
SEQ ID NO: 14
Met Ala Thr Gly Gly Arg Arg Gly Ala Ala Ala Ala
  1 5 10
Pro Leu Leu Val Ala Val Ala Ala Leu Leu Leu Gly
                  15 16 20
Ala Ala Gly His Leu Tyr Pro Gly Glu Val Cys Pro
  25 30 31 35
Gly Met Asp Ile Arg Asn Asn Leu Thr Arg Leu His
                          40 45 46
Glu Leu Glu Asn Cys Ser Val Ile Glu Gly His Leu
          50 55 60
Gln Ile Leu Leu Met Phe Lys Thr Arg Pro Glu Asp
61 65 70
Phe Arg Asp Leu Ser Phe Pro Lys Leu Ile Met Ile
                  75 76 80
Thr Asp Tyr Leu Leu Leu Phe Arg Val Tyr Gly Leu
85 90 91 95
Glu Ser Leu Lys Asp Leu Phe Pro Asn Leu Thr Val
                      100 105 106
Ile Arg Gly Ser Arg Leu Phe Phe Asn Tyr Ala Leu
        110 115 120
Val Ile Phe Glu Met Val His Leu Lys Glu Leu Gly
121 125 130
Leu Tyr Asn Leu Met Asn Ile Thr Arg Gly Ser Val
                135 136 140
Arg Ile Glu Lys Asn Asn Glu Leu Cys Tyr Leu Ala
145 150 151 155
Thr Ile Asp Trp Ser Arg Ile Leu Asp Ser Val Glu
                        160 165 166
Asp Asn Tyr Ile Val Leu Asn Lys Asp Asp Asn Glu
        170 175 180
Glu Cys Gly Asp Ile Cys Pro Gly Thr Ala Lys Gly
181 185 190
Lys Thr Asn Cys Pro Ala Thr Val Ile Asn Gly Gln
                195 196 200
Phe Val Glu Arg Cys Trp Thr His Ser His Cys Gln
205 210 211 215
Lys Val Cys Pro Thr Ile Cys Lys Ser His Gly Cys
                        220 225 226
Thr Ala Glu Gly Leu Cys Cys His Ser Glu Cys Leu
        230 235 240
Gly Asn Cys Ser Gln Pro Asp Asp Pro Thr Lys Cys
241 245 250
Val Ala Cys Arg Asn Phe Tyr Leu Asp Gly Arg Cys
                255 256 260
Val Glu Thr Cys Pro Pro Pro Tyr Tyr His Phe Gln
265 270 271 275
Asp Trp Arg Cys Val Asn Phe Ser Phe Cys Gln Asp
                        280 285 286
Leu His His Lys Cys Lys Asn Ser Arg Arg Gln Gly
        290 295 300
Cys His Gln Tyr Val Ile His Asn Asn Lys Cys Ile
301 305 310
Pro Glu Cys Pro Ser Gly Tyr Thr Met Asn Ser Ser
                315 316 320
Asn Leu Leu Cys Thr Pro Cys Leu Gly Pro Cys Pro
325 330 331 335
Lys Val Cys His Leu Leu Glu Gly Glu Lys Thr Ile
                        340 345 346
Asp Ser Val Thr Ser Ala Gln Glu Leu Arg Gly Cys
        350 355 360
Thr Val Ile Asn Gly Ser Leu Ile Ile Asn Ile Arg
361 365 370
Gly Gly Asn Asn Leu Ala Ala Glu Leu Glu Ala Asn
                375 376 380
Leu Gly Leu Ile Glu Glu Ile Ser Gly Tyr Leu Lys
385 390 391 395
Ile Arg Arg Ser Tyr Ala Leu Val Ser Leu Ser Phe
                        400 405 406
Phe Arg Lys Leu Arg Leu Ile Arg Gly Glu Thr Leu
        410 415 420
Glu Ile Gly Asn Tyr Ser Phe Tyr Ala Leu Asp Asn
421 425 430
Gln Asn Leu Arg Gln Leu Trp Asp Trp Ser Lys His
                435 436 440
Asn Leu Thr Ile Thr Gln Gly Lys Leu Phe Phe His
445 450 451 455
Tyr Asn Pro Lys Leu Cys Leu Ser Glu Ile His Lys
                        460 465 466
Met Glu Glu Val Ser Gly Thr Lys Gly Arg Gln Glu
        470 475 480
Arg Asn Asp Ile Ala Leu Lys Thr Asn Gly Asp Gln
481 485 490
Ala Ser Cys Glu Asn Glu Leu Leu Lys Phe Ser Tyr
                495 496 500
Ile Arg Thr Ser Phe Asp Lys Ile Leu Leu Arg Trp
505 510 511 515
Glu Pro Tyr Trp Pro Pro Asp Phe Arg Asp Leu Leu
                        510 525 526
Gly Phe Met Leu Phe Tyr Lys Glu Ala Pro Tyr Gln
        530 535 540
Asn Val Thr Glu Phe Asp Gly Gln Asp Ala Cys Gly
541 545 550
Ser Asn Ser Trp Thr Val Val Asp Ile Asp Pro Pro
                555 556 560
Leu Arg Ser Asn Asp Pro Lys Ser Gln Asn His Pro
565 570 571 575
Gly Trp Leu Met Arg Gly Leu Lys Pro Trp Thr Gln
                        580 585 586
Tyr Ala Ile Phe Val Lys Thr Leu Val Thr Phe Ser
        590 595 600
Asp Glu Arg Arg Thr Tyr Gly Ala Lys Ser Asp Ile
601 605 610
Ile Tyr Val Gln Thr Asp Ala Thr Asn Pro Ser Val
                615 616 620
Pro Leu Asp Pro Ile Ser Val Ser Asn Ser Ser Ser
625 630 631 635
Gln Ile Ile Leu Lys Trp Lys Pro Pro Ser Asp Pro
                        640 645 646
Asn Gly Asn Ile Thr His Tyr Leu Val Phe Trp Glu
        650 655 660
Arg Gln Ala Glu Asp Ser Glu Leu Phe Glu Leu Asp
661 665 670
Tyr Cys Leu Lys Gly Leu Lys Leu Pro Ser Arg Thr
                675 676 680
Trp Ser Pro Pro Phe Glu Ser Glu Asp Ser Gln Lys
685 690 691 695
His Asn Gln Ser Glu Tyr Glu Asp Ser Ala Gly Glu
                        700 705 706
Cys Cys Ser Cys Pro Lys Thr Asp Ser Gln Ile Leu
        710 715 720
Lys Glu Leu Glu Glu Ser Ser Phe Arg Lys Thr Phe
721 725 730
Glu Asp Tyr Leu His Asn Val Val Phe Val Pro Arg
                735 736 740
Lys Thr Ser Ser Gly Thr Gly Ala Glu Asp Pro Arg
745 750 751 755
Pro Ser Arg Lys Arg Arg Ser Leu Gly Asp Val Gly
                        760 765 766
Asn Val Thr Val Ala Val Pro Thr Val Ala Ala Phe
        770 775 780
Pro Asn Thr Ser Ser Thr Ser Val Pro Thr Ser Pro
781 785 790
Glu Glu His Arg Pro Phe Glu Lys Val Val Asn Lys
                795 796 800
Glu Ser Leu Val Ile Ser Gly Leu Arg His Phe Thr
805 810 811 815
Gly Tyr Arg Ile Glu Leu Gln Ala Cys Asn Gln Asp
                        820 825 826
Thr Pro Glu Glu Arg Cys Ser Val Ala Ala Tyr Val
        830 835 840
Ser Ala Arg Thr Met Pro Glu Ala Lys Ala Asp Asp
841 845 850
Ile Val Gly Pro Val Thr His Glu Ile Phe Glu Asn
                855 856 860
Asn Val Val His Leu Met Trp Gln Glu Pro Lys Glu
865 870 871 875
Pro Asn Gly Leu Ile Val Leu Tyr Glu Val Ser Tyr
                        880 885 886
Arg Arg Tyr Gly Asp Glu Glu Leu His Leu Cys Val
        890 895 900
Ser Arg Lys His Phe Ala Leu Glu Arg Gly Cys Arg
901 905 910
Leu Arg Gly Leu Ser Pro Gly Asn Tyr Ser Val Arg
                915 916 920
Ile Arg Ala Thr Ser Leu Ala Gly Asn Gly Ser Trp
925 930 931 935
Thr Glu Pro Thr Tyr Phe Tyr Val Thr Asp Tyr Leu
                        940 945 946
Asp Val Pro Ser Asn Ile Ala Lys Ile Ile Ile Gly
        950 955 960
Pro Leu Ile Phe Val Phe Leu Phe Ser Val Val Ile
961 965 970
Gly Ser Ile Tyr Leu Phe Leu Arg Lys Arg Gln Pro
                975 976 980
Asp Gly Pro Leu Gly Pro Leu Tyr Ala Ser Ser Asn
985 990 991 995
Pro Glu Tyr Leu Ser Ala Ser Asp Val Phe Pro Cys
                      1000 1005 1006
Ser Val Tyr Val Pro Asp Glu Trp Glu Val Ser Arg
      1010 1015 1020
Glu Lys Ile Thr Leu Leu Arg Glu Leu Gly Gln Gly
1021 1025 1030
Ser Phe Gly Met Val Tyr Glu Gly Asn Ala Arg Asp
              1035 1036 1140
  Ile Ile Lys Gly Glu Ala Glu Thr Arg Val Ala Val
1145 1050 1051 1155
Lys Thr Val Asn Glu Ser Ala Ser Leu Arg Glu Arg
                      1160 1065 1066
Ile Glu Phe Leu Asn Glu Ala Ser Val Met Lys Gly
      1170 1175 1080
Phe Thr Cys His His Val Val Arg Leu Leu Gly Val
1081 1185 1190
Val Ser Lys Gly Gln Pro Thr Leu Val Val Met Glu
              1095 1096 1100
  Leu Met Ala His Gly Asp Leu Lys Ser Tyr Leu Arg
1105 1110 1111 1115
Ser Leu Arg Pro Glu Ala Glu Asn Asn Pro Gly Arg
                      1120 1125 1126
Pro Pro Pro Thr Leu Gln Glu Met Ile Gln Met Ala
      1130 1135 1140
Ala Glu Ile Ala Asp Gly Met Ala Tyr Leu Asn Ala
1141 1145 1150
Lys Lys Phe Val His Arg Asp Leu Ala Ala Arg Asn
              1155 1156 1160
  Cys Met Val Ala His Asp Phe Thr Val Lys Ile Gly
1165 1170 1171 1175
Asp Phe Gly Met Thr Arg Asp Ile Tyr Glu Thr Asp
                      1180 1185 1186
Tyr Tyr Arg Lys Gly Gly Lys Gly Leu Leu Pro Val
      1190 1195 1200
Arg Trp Met Ala Pro Glu Ser Leu Lys Asp Gly Val
1201 1205 1210
Phe Thr Thr Ser Ser Asp Met Trp Ser Phe Gly Val
              1215 1216 1220
  Val Leu Trp Glu Ile Thr Ser Leu Ala Glu Gln Pro
1225 1230 1231 1235
Tyr Gln Gly Leu Ser Asn Glu Gln Val Leu Lys Phe
                      1240 1245 1246
Val Met Asp Gly Gly Tyr Leu Asp Gln Pro Asp Asn
      1250 1255 1260
Cys Pro Glu Arg Val Thr Asp Leu Met Arg Met Cys
1261 1265 1270
Trp Gln Phe Asn Pro Lys Met Arg Pro Thr Phe Leu
              1275 1276 1280
  Glu Ile Val Asn Leu Leu Lys Asp Asp Leu His Pro
1285 1290 1291 1295
Ser Phe Pro Glu Val Ser Phe Phe His Ser Glu Glu
                      1300 1305 1306
Asn Lys Ala Pro Glu Ser Glu Glu Leu Glu Met Glu
      1310 1315 1320
Phe Glu Asp Met Glu Asn Val Pro Leu Asp Arg Ser
1321 1325 1330
Ser His Cys Gln Arg Glu Glu Ala Gly Gly Arg Asp
              1335 1336 1340
  Gly Gly Ser Ser Leu Gly Phe Lys Arg Ser Tyr Glu
1345 1350 1351 1355
Glu His Ile Pro Tyr Thr His Met Asn Gly Gly Lys
                      1360 1365 1366
Lys Asn Gly Arg Ile Leu Thr Leu Pro Arg Ser Asn
      1370 1375 1380
Pro Ser
13811382
```

The exemplary procedure for preparation of the starting polyclonal antibodies to C-terminal fragment of beta subunit of human insulin receptor may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations. Supernatant fluid is the target antiserum. The obtained antiserum is typically yellow. 20% of NaN₃ (weight concentration) is added in the antiserum to the final concentration of 0.02% and stored before use in frozen state at the temperature of −20° C. (or without NaN₃ at the temperature of −70° C.). To separate the target antibodies to C-terminal fragment of beta subunit of human insulin-receptor from the antiserum, the following solid phase absorption sequence is suitable:

10 ml of the antiserum of rabbits is diluted twofold with 0.15 M NaCl, after which 6.26 g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to DEAE-cellulose column balanced by phosphate buffer. The antibody fraction is determined by measuring the optical density of eluate at 280 Nm.

The isolated crude antibodies are purified using the affine chromatography method by attaching the obtained antibodies to a C-terminal fragment of beta subunit of human insulin receptor located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to C-terminal fragment of beta subunit of human insulin-receptor is 0.5 to 5.0 mg/ml, preferably, 2.0 to 3.0 mg/ml.

The activated potentiated form may be prepared from initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to C-terminal fragment of beta subunit of human insulin receptor with the concentration of 3.0 mg/ml is diluted in 99 parts of neutral aqueous or aqueous-alcohol solvent (preferably, 15%-ethyl alcohol) and then vertically shaken many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to C-terminal fragment of beta subunit of human insulin-receptor with the concentration of 3.0 mg/ml in 99 parts of a neutral solvent in different containers, which is equivalent to the centesimal homeopathic dilution C12. Similar procedures with the relevant dilution factor are performed to obtain dilutions C30 and C 200. The intermediate dilutions may be tested in a desired biological model to check activity. The preferred activated potentiated forms for both antibodies comprising the combination of the invention are a mixture of C12, C30, and C200 dilutions. When using the mixture of various homeopathic dilutions (primarily centesimal) of the active substance as biologically active liquid component, each component of the composition (e.g., C12, C30, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D 20, C 30, C100 or C12, C30, C50 etc.), the efficiency of which is determined experimentally by testing the dilution in a suitable biological model, for example, in models described in the examples herein.

In course of potentiation and concentration decrease, the vertical shaking may be substituted for external exposure to ultrasound, electromagnetic field or any similar external impact procedure accepted in the homeopathic art.

Preferably, the pharmaceutical composition of the invention may be in the form of a liquid or in the solid unit dosage form. The preferred liquid form of the pharmaceutical composition is a mixture, preferably, at a 1:1 ratio of the activated potentiated form of antibodies. The preferred liquid carrier is water or water-ethyl alcohol mixture.

The solid unit dosage form of the pharmaceutical composition of the invention may be prepared by impregnating a solid, pharmaceutically acceptable carrier with the mixture of the activated potentiated form of aqueous or aqueous-alcohol solutions of active components. Alternatively, the carrier may be impregnated consecutively with each requisite dilution. Both orders of impregnation are acceptable.

Preferably, to prepare the claimed veterinary composition in a form of a compound drug, the aqueous or aqueous-alcoholic solutions of the active components are mixed (primarily in 1:1:1 ratio by volume) and used in a liquid dosage form.

The veterinary composition of the invention may also be in a solid unit dosage form (formulated as a powder or tablet) and represent a compound drug containing a technologically required (efficient) amount of a neutral carrier (e.g. lactose) saturated by impregnation with, for example, a mixture of aqueous or aqueous-alcohol solutions of the activated-potentiated form of antibodies to the insulin receptor β-subunit (antibodies to a C-terminal fragment of the insulin receptor β-subunit) in combination with pharmaceutically acceptable excipients, primarily including lactose, microcrystalline cellulose and magnesium stearate.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies to C-terminal fragment of beta subunit of human insulin-receptor. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono-olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose and magnesium stearate.

To prepare the solid oral form formulated as a tablet, 50-500 μm granules of the neutral excipient—lactose (milk sugar), which were previously saturated with an aqueous or aqueous-alcoholic solution of the activated-potentiated form of antibodies to the insulin receptor β-subunit (or, for example, antibodies to insulin receptor β-subunit, to human interferon gamma, and to CD4 receptor) in the ratio of 1 kg of antibody solution to 5 or 10 kg of lactose (1:5 to 1:10), are exposed to saturation irrigation in the fluidized boiling bed in a fluid bed system (e.g. “Hütlin Pilotlab” by Hütlin GmbH) with subsequent drying with preheated air flow introduced through the bed plate at a temperature below 40° C. The estimated amount of the lactose (10÷91% of the tablet mass (by weight)) saturated with the activated-potentiated form of antibodies according to the above-described processing procedure is loaded in the mixer hopper, and mixed with lactose saturated with the activated-potentiated form of antibodies taken at the amount of 3 to 10 weight parts (3÷10% of the tablet mass) and with no more than 84 weight parts (81% of the tablet mass) of “non-saturated” pure lactose (used for the purposes of cost reduction and simplification and acceleration of the technological process without decreasing the treatment efficiency). Then the mixture is supplemented with 5 to 10 weight parts (5÷10% of the tablet mass) of cellulose and 1 weight part 1% of the tablet mass) of magnesium stearate. The obtained tablet mass is uniformly mixed, and tableted by direct dry pressing (e.g., in a Korsch—XL 400 tablet press) to form 150 to 500 mg round pills. After tableting, 300 mg pills are obtained that are saturated with aqueous-alcoholic solution (3.0-6.0 mg/pill) of the activated-potentiated form of antibodies to the insulin receptor β-subunit. The component used to impregnate the carrier is in an ultra-low dose prepared from the initial matrix solution diluted by a factor of 100¹², 100³⁰ and 100⁵⁰, which is equivalent to a mixture of centesimal homeopathic dilutions C12, C30 and C50.

While the invention is not limited to any specific theory, it is believed that the activated potentiated form of the antibodies described herein do not contain the molecular form of the antibody in the amount sufficient to have biological activity attributed to such molecular form. The biological activity of the composition of the invention is amply demonstrated in the appended examples.

The composition of the invention may be used for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

### EXAMPLES

### Example 1

The effect of the claimed compound intended for promoting body weight gain in mammals and birds, enhancing the effectiveness of immunization, and preventing and/or treating infectious diseases, in the form of aqueous solution containing an activated-potentiated form of antigen-purified ultra-low dose polyclonal rabbit antibodies to the insulin receptor n-subunit (prepared by extreme dilution of the primary matrix solution (concentration of 2.5 mg/ml) by a factor of 100¹², 100³⁰, 100²⁰⁰), which is equivalent to a mixture of centesimal homeopathic C12, C30 and C200 dilutions (anti-IRβ Ab), on body weight changes was evaluated in mature male albino Wistar rats ( ). The test compound was administered intragastrically (via a gavage needle) at 2.5 ml/kg once daily for 6 months (n=20). The control animals were dosed in a similar manner with 2.5 ml/kg of settled water (n=20). The overall study duration, including a period of one month after treatment discontinuation, was 7 months. General health and body weight changes of the animals were recorded regularly at monthly intervals.

There were no differences in general health assessments between the animal groups throughout the study period: the animals did not show restlessness or changes in appetite, defecation, and state of the mucosa, hair and skin, etc. Body weight data at different monitoring time points are summarized in Table 1. There was an increase (p>0.05) observed as soon as at the end of the second month of monitoring in the weight gain values of animals receiving RA anti-IRβ Ab compared to the control group. At 3, 4, 5 and 6 months of the dosing period, the rats' body weights were significantly incremented in the RA anti-IRβ group as compared to control animals. The noted body weight increases as related to the control group were 6.1%, 9.4%, 10.4% and 11.2% at 3, 4, 5 and 6 months of the dosing period, respectively. Following one month after treatment discontinuation, the rats' body weights in the RA anti-IRβ group remained increased as compared to control values (p>0.05).

TABLE 1
Body weight changes of male Wistar rats

| | Control | anti-IRβ Ab |
|---|---|---|
| Month 1 | 216.75 ± 3.96 | 204.75 ± 5.05 |
| Month 2 | 233.75 ± 4.23 | 242.00 ± 3.72 |
| Month 3 | 256.50 ± 4.11 | 272.25 ± 4.3* |
| Month 4 | 269.00 ± 4.24 | 294.21 ± 3.98* |
| Month 5 | 280.00 ± 3.72 | 309.21 ± 4.64* |
| Month 6 | 289.25 ± 3.43 | 321.58 ± 4.81* |
| Month 1 | 318.33 ± 6.94 | 331.82 ± 6.75 |
| post-discontinuation | | |

*p > 0.05 compared to controls

What is claimed is:

1. A method of improving livability of food-producing animals, non-human mammals or birds, said method comprising administering to said animal, non-human mammal or bird an activated-potentiated form of an antibody to the insulin receptor β-subunit.

2. The method of claim 1 comprising administering an activated-potentiated form of an antibody to a C-terminal fragment of the insulin receptor β-subunit.

3. The method of claim 2, wherein said activated-potentiated form of an antibody to a C-terminal fragment of the insulin receptor β-subunit is in the form of an aqueous or aqueous-alcoholic solution with the activity achieved through repeated sequential dilution of the primary matrix solution of an antibody to a C-terminal fragment of the insulin receptor β-subunit in a water or alcohol-water solvent, coupled with external mechanical treatment of each dilution.

4. The method of claim 1, wherein a mixture of homeopathic dilutions of an antibody to a C-terminal fragment of the insulin receptor β-subunit is used as a unit dosage form.

5. A method of promoting body weight gain in non-human mammals or birds, said method comprising administering to said non-human mammal or bird an activated-potentiated form of an antibody to the insulin receptor β-subunit.

6. The method of claim 5 comprising administering to the animal an activated-potentiated form of an antibody to a C-terminal fragment of the insulin receptor β-subunit.

7. The method of claim 6, wherein said activated-potentiated form of an antibody to a C-terminal fragment of the insulin receptor β-subunit is in the form of an aqueous or aqueous-alcoholic solution with the activity achieved through repeated sequential dilution of the primary matrix solution of antibody to a C-terminal fragment of the insulin receptor β-subunit in a water or alcohol-water solvent, coupled with external mechanical treatment of each dilution.

8. The method of claim 6, wherein a mixture of homeopathic dilutions of an antibody to a C-terminal fragment of the insulin receptor β-subunit is used as a unit dosage form.

9. A method of enhancing the effectiveness of immunization in mammals or birds, said method comprising administering to said mammal or bird an activated-potentiated form of an antibody to the insulin receptor β-subunit.

10. The method of claim 9, said method comprising administering to the animal an activated-potentiated form of an antibody to a C-terminal fragment of the insulin receptor β-subunit.

11. The method of claim 10, wherein said activated-potentiated form of an antibody to a C-terminal fragment of the insulin receptor β-subunit is in the form of an aqueous or aqueous-alcoholic solution with the activity achieved through repeated sequential dilution of the primary matrix solution of antibody to a C-terminal fragment of the insulin receptor β-subunit in a water or alcohol-water solvent, coupled with external mechanical treatment of each dilution.

12. The method of claim 9, wherein a mixture of homeopathic dilutions of an antibody to a C-terminal fragment of the insulin receptor β-subunit is used as a unit dosage form.

13. A method of preventing and/or treating infectious diseases of non-human mammals or birds, said method comprising administering to said non-human mammal or bird an activated-potentiated form of an antibody to the insulin receptor β-subunit.

14. The method of claim 13 comprising administering to the animal an activated-potentiated form of an antibody to a C-terminal fragment of the insulin receptor β-subunit.

15. The method of claim 14 wherein said activated-potentiated form of an antibody to a C-terminal fragment of the insulin receptor β-subunit is in the form of an aqueous or aqueous-alcoholic solution with the activity achieved through repeated sequential dilution of the primary (matrix) solution of antibody to a C-terminal fragment of the insulin receptor β-subunit in a water or alcohol-water solvent, coupled with external mechanical treatment of each dilution.

16. The method of claim 14, wherein a mixture of various homeopathic dilutions of an antibody to a C-terminal fragment of the insulin receptor β-subunit is used as a unit dosage form.
